(19) United States
(12) Patent Application Publication
(10) Pub. No.: US 2012/0128639 A1
(43) Pub. Date: May 24, 2012

(54) In Vitro Production Of Oligodendrocytes From Human Umbilical Cord Stem Cells

(75) Inventors: James J. Hickman, Orlando, FL (US); Hedvika Davis, Orlando, FL (US)

(73) Assignee: UNIVERSITY OF CENTRAL FLORIDA RESEARCH FOUNDATION, INC., Orlando (FL)

(21) Appl. No.: 13/322,903

(22) PCT Filed: May 28, 2010
(86) PCT No.: PCT/US10/36505

§ 371 Date: Feb. 9, 2012

Related U.S. Application Data
(60) Provisional application No. 61/181,868, filed on May 28, 2009.

(51) Int. Cl. A61K 35/30; A61P 25/00; C12N 5/079

(52) U.S. Cl. 424/93.7; 435/366

(57) ABSTRACT

The invention provides a method of producing oligodendrocytes by in vitro differentiation of human multi-potent progenitor cells (MLPCs). The method comprises culturing isolated MLPCs on a first surface in a serum-free defined culture medium; replacing the culture medium with serum-free culture medium supplemented with bFGF, EGF and PDGF-AA for approximately 24 hours; changing the cultured MLPCs into the supplemented serum-free culture medium further supplemented with differentiation factors norepinephrine, forskolin. and K252a; establishing a 3D environment by covering the culture with a second surface opposite and spaced apart from the first surface, so as to contain the MLPCs therebetween; and continuing to culture until a majority of the MLPCs have differentiated into oligodendrocytes. Additionally included is a method of treatment for a subject afflicted by a disease characterized by central or peripheral nervous system demyelination, the method comprising transplanting into the subject oligodendrocytes produced according to the method disclosed.

### RELATED APPLICATION

This application claims priority from co-pending provisional application Ser. No. 61/181,868, which was filed on 28 May 2009; and which is incorporated herein by reference in its entirety.

### STATEMENT OF GOVERNMENT RIGHTS

The invention claimed herein was made with at least partial support from the U.S. Government. Accordingly, the government may have certain rights in the invention, as specified by law.

### FIELD OF THE INVENTION

The present invention relates to the field of stem cells and, more particularly, to the differentiation of multipotent progenitor cells (MLPC) from umbilical cord into oligodendrocytes in a three-dimensional (3D) in vitro environment.

### BACKGROUND OF THE INVENTION

Differentiation of oligodendrocytes in the local tissue environment depends on gradients of soluble factors and physical cues that activate distinct signaling pathways. One of the soluble factors is norepinephrine (NE), a small molecule neurotransmitter released from noradrenergic neurons. The effect of NE on oligodendrocyte differentiation is not yet well understood. However, it has been reported that noradrenergic fibers contact oligodendrocytes at sites that resemble symmetrical synapses, suggesting that oligodendrocytes could be NE's primary target (Paspalas and Papadopoulos, 1996). It also has been determined that NE binds to and activates α and β-adrenergic receptors (ARs), and oligodendrocytes express both α-1 and β-ARs (Ghiani et al., 1999; Khorchid et al., 2002; Khorchid et al., 1999: Ventimiglia et al., 1987). It has been shown that activation of α-1 adrenergic signaling influences the formation of processes and the production of myelin. NE has also been shown to increase the activity of protein kinase C (PKC), p38 mitogen-activated protein kinases (MAPK) and phosphoinositide (PI) hydrolysis (Asotra and Macklin, 1993; Cohen and Almazan, 1993; Khorchid et al. 2002; Khorchid et al., 1999). Alternatively, activation of β-adrenergic signaling by NE inhibits proliferation and accelerated lineage progression (Ghiani et al., 1999). The biological effect of NE was found to be mediated through an increase in intracellular cAMP, activity of ERK (extracellular signal-regulated kinase) and of proteins essential for cell cycle arrest (Bernstein et al., 1996; Ghiani et al., 1999; Vartanian et al., 1988). It was suggested that β-AR mediated signaling may be restricted to the proliferative phases of oligodendrocyte development, and dismantled after proliferation arrest.

The physical cues that modify differentiation are defined by mechanical forces and discrete local architecture (Burdick and Vunjak-Novakovic, 2008; Vogel and Sheetz, 2006). 3D environments have been shown to be especially important in tissue engineering applications (Bettinger et al., 2009; Fisher et al., 2009: Levenberg et al., 2003; Luo et al., 2006). These conserved and evolutionary ancient features, although largely unknown, may be the source of many developmental cues. During embryogenesis, signals are transduced to a stem or progenitor cell's nuclei through changes in the cytoskeleton and through complex signaling pathways. For example, it has been shown that the fate decision of oligodendrocyte precursors is controlled by both spatial and geometric characteristics of an axonal niche (Rosenberg et al., 2008). The critical cell density along the axon provides a mechanical stimulus that promotes differentiation, possibly through alteration of the size or shape of the developing oligodendrocytes. Another example presented studies with mesenchymal stem cells (MSCs) where it was demonstrated that the size of the substrate pattern regulated cell shape and the resultant cytoskeletal tension, which controlled the lineage commitment (McBeath et al., 2004). Stem cell fate has also been shown to be directed by the elasticity of the matrix, which is key to controlling variables in the in vivo tissue environment (Engler et al., 2006). We have previously demonstrated how physical as well as chemical cues control the function of endothelial and neuronal cells in a defined system. In these studies, chemically defined surfaces and media were used to direct cell adhesion, spreading and differentiation (Das et al., 2005; Schaffner et al., 1995; Spargo et al., 1994; Stenger et al., 1993; Varghese et al., 2009).

### SUMMARY OF THE INVENTION

During differentiation stem cells are exposed to a range of microenvironmental chemical and physical cues. In this study, we differentiated human multipotent progenitor cells (MLPCs) from umbilical cord into oligodendrocytes. Chemical cues were represented by a novel defined differentiation medium containing the neurotransmitter norepinephrine (NE). In traditional 2 dimensional (2D) conditions, the MLPCs differentiated into oligodendrocyte precursors, but did not progress further. However, in a 3 dimensional (3D) environment, the MLPCs differentiated into committed oligodendrocytes that expressed MBP. This study presents a novel method of obtaining oligodendrocytes from human MLPCs that could eliminate many of the difficulties associated with their differentiation from embryonic stem cells. In addition, it reveals the complex interplay between physical cues and biomolecules on stem cell differentiation.

With the foregoing in mind, the present invention advantageously provides human multipotent progenitor cells differentiated into oligodendrocytes, where induction is promoted by norepinephrin in a serum-free, defined in vitro system. A 3D environment is necessary for complete differentiation and MBP expression, and action of both the α-1 and β adrenergic receptors is necessary.

This disclosure represents the first example of small molecule induction of oligodendrocytes from stem cells. The multipotent progenitor cells (MLPCs) obtained from human umbilical cord were differentiated into oligodendrocytes in the presence of norepinephrine (NE) in a 3D environment. Differentiation of these cells in a 2D environment was not sufficient to enable complete functional maturation. Oligodendrocytes, the cells that produce myelin and maintain myelination of axons in the central nervous system (CNS), originate early during embryogenesis (Bunge et al., 1962; Bunge, 1968; Hirano, 1968; Kessaris et al., 2008: Orentas and Miller, 1998; Peters, 1964). These cells derive from neuroepithelial stem cells during development and give rise to first neuronal and later glial progenitor cells. Glial progenitors migrate, divide and terminally differentiate into astrocytes, microglia and oligodendrocytes (Kessaris et al., 2008; LeVine and Goldman, 1988; Noll and Miller, 1993; Warf et al., 1991). The progression of the oligodendroglial lineage is characterized by dramatic morphological changes and acquisition of specific surface antigens (Bansal and Pfeiffer, 1992; Behar, 2001; Curtis et al., 1988; Pfeiffer et al., 1993; Sternberger et al., 1985; Volpe, 2008). The oligodendrocyte progenitors can be detected with the A2B5 antibody followed by the expression of the O4 sulfatide, which persists in ramified, but yet immature oligodendrocytes. Committed oligodendrocytes lose A2B5 reactivity after they begin to express O1 galactocerebroside. Differentiated oligodendrocytes, which are post-mitotic and richly multipolar cells, express myelin basic protein (MBP) upon maturation and gradually initiate the myelination of neurons in the CNS.

In the present investigation we constructed a 3D environment that guided differentiation of human MLPCs from umbilical cord into mature oligodendrocytes. This 3D environment provided an optimal combination of chemical and physical cues where all the parameters were known and controllable. The chemical cues were represented by a number of soluble factors of which NE played the key stimulating function through the α-1 and β adrenergic receptors. The soluble factors alone in the standard 2D conditions were able to induce differentiation of MLPCs along the initial stages of oligodendrocyte lineage; however, differentiation of the MLPCs into oligodendrocytes was achieved only in 3D conditions. To date, human oligodendrocytes have been produced from embryonic or fetal stem cells, raising ethical considerations (Hu et al., 2009; Izrael et al. 2007; Liu et al., 2000; Nistor et al., 2005; Rogister et al., 1999). MLPCs, unlike embryonic stem cells (ESCs), do not spontaneously differentiate in vitro, yet they are capable of extensive differentiation and expansion (van de Ven et al., 2007).

Differentiation of MLPCs in vitro according to the present invention could generate unlimited numbers of oligodendrocytes for studies of various differentiation stages or for transplantation to treat demyelinating diseases, such as multiple sclerosis. From a technological standpoint, this would be advantageous as the time to differentiate is much less for the MLPCs than for ESCs and also MLPCs can be induced using small molecules, without genetic manipulation, in a defined, serum free system.

Accordingly, the invention provides a method of producing oligodendrocytes by in vitro differentiation of human multi-potent progenitor cells (MLPCs). The method includes culturing isolated MLPCs on a first surface in a serum-free defined culture medium; replacing the culture medium with serum-free culture medium supplemented with bFGF. EGF and PDGF-AA for approximately 24 hours; establishing a 3D environment by covering the culture with a second surface opposite and spaced apart from the first surface, so as to contain the MLPCs therebetween; changing the cultured MLPCs into the supplemented serum-free culture medium further supplemented with differentiation factors norepinephrine, forskolin, and K252a: and continuing to culture until a majority of the MLPCs have differentiated into oligodendrocytes.

In the method, the first surface preferably comprises a pre-treated sterile surface and, more specifically, a DETA-coated glass surface. Also, in the method culturing is preferably continued until the MLPCs reach approximately 60% confluence. In one preferred embodiment of the method establishing the 3D environment is concurrent with the replacing step where medium is changed. Thos of skill in the art will recognize that the invention includes oligodendrocytes produced according to any of the culture methods described herein. The produced oligodendrocytes may be used in a method of treatment for a subject afflicted by a disease characterized by central or peripheral nervous system deficit by transplanting into the subject the oligodendrocytes produced. In a preferred treatment method, the deficit comprises demyelination of the nervous system, whether central or peripheral.

More broadly, the method of the present invention is useful for producing oligodendrocytes in vitro by culturing human MLPCs within a three-dimensional environment in a defined serum-free growth medium and sufficiently stimulating adrenergic pathways in the MLPCs so as to induce their differentiation into oligodendrocytes.

### BRIEF DESCRIPTION OF THE DRAWINGS

Some of the features, advantages, and benefits of the present invention having been stated, others will become apparent as the description proceeds when taken in conjunction with the accompanying drawings in which:

FIG. 1 shows an immunocytochemical analysis of untreated MLPC suggest neuroepithelial origin; untreated MLPCs expressed the neuroepithelial marker Sox-1, stained positively for PDGFR-α, PDGFR-β and negatively for A2B5; scale bar, 100 μm, (200× magnification);

FIG. 2 depicts phase contrast images of differentiating MLPCs in 2D Environment; (A) undifferentiated MLPC exhibited fibroblast morphology; (B-H) cells at 15 days in differentiation medium without: (B) NE, (C) forskolin or (D) K252a, retained their fibroblast morphology; (E) process formation was visible in the medium without growth factors; (F-H) refractile cell bodies and increased process formation were observed in the presence of all factors indicated above; (I) cells lost their multipolar morphology and became bipolar or spindle shaped at day 24; scale bars, 100 μm, (panels A-F, 1,200× magnification, panels G-H, 400× magnification);

FIG. 3 illustrates how a 2D Environment promotes differentiation of MLPCs along early stages of oligodendroglial lineage; (A) immunocytochemical analysis of differentiating MLPC in 2D environment; the untreated MLPCs showed negative staining for A2B5 and faint staining for O4; at 15 days of differentiation, cells exhibited positive staining for A2B5 and O4, characteristics of immature oligodendrocyte precursor cells; (B) MLPC do not differentiate into committed oligodendrocytes in 2D Environment; at 8 days of differentiation, 72.4% of cells were positive for A2B5 and 69.9% for O4 and at day fifteen 70.3% of MLPC exhibited positive staining for A2B5 and 69.7% for O4; at 20 days, 35.0% of cells remained A2B5 positive and 49.7% O4 positive; expression of O1 galactocerebroside and MBP was absent in both untreated and differentiating cells; scale bar, 100 μm, (Rows 1 and 2, 200× magnification and Row 3, 400× magnification); error bars represent the SD.

FIG. 4 shows how a 3D Environment promotes further differentiation of MLPCs: (A-F) Phase contrast images of differentiating MLPCs; (A) the untreated cells displayed typical fibroblast morphology; (B) cells exhibited flattening and spreading at 24 hrs in 3D environment; (C) cells at 8 days in the differentiation medium displayed increased flattening; (D, E, F) at 30 days of differentiation, approximately 80% of cells revealed extensive processes; (G-I) growth factors influenced the development of processes; (G, H) immunostained cells displaying increased branching and development of processes in presence of bFGF and EGF; (I) simple processes were observed in absence bFGF and EGF; scale bars, 100 μm, (400× magnification);

FIG. 5 depicts how MLPCs differentiate into committed oligodendrocytes in a 3D Environment; (A) immunocytochemical analysis of differentiating MLPCs in a 3D environment; the untreated MLPCs indicated negative staining for A2B5, faint staining for O4 and negative staining for O1 galactocerebroside and MBP: at 30 days of differentiation, cells exhibited intensely positive staining for A2B5 and O4; cells also expressed O1 galactocerebroside and MBP, characteristic of committed oligodendrocytes; scale bars, 100 μm, (Rows 1-7, 200× magnification and Rows 4-5, 400× magnification); (B) co-expression of O4 and galactocerebroside (GC) in the differentiated cells; at 30 days of differentiation, GC was expressed in O4 positive cells; scale bars, 100 μm, (Row 1, 200× magnification and Row 2, 400× magnification); (C) the progression of differentiation in a 3D environment; at 20 days of differentiation 81.8% of cells expressed oligodendroglial markers A2B5 and 80.6% O4 and were negative for O1 and MBP; at 30 days of differentiation, 57.7% of cells stained positively for A2B5, 79.6% for O4, 42.1% for committed oligodendrocyte marker O1 and 15.2% for MBP; error bars represent the SD;

FIG. 6 shows expression of α1- and β1-ARs; (A) positive staining for β1-ARs in undifferentiated cells, cells pre-induced for 24 hrs with bFGF, EGF and PDGF-AA, and cells at 30 days of differentiation; scale bar, 100 μm; (B) nuclear expression of α1-ARs in undifferentiated cells, intensive nuclear staining in cells pre-induced for 24 hrs with bFGF, EGF and PDGF-AA, and surface expression at 30 days of differentiation; (C) nuclear expression of α1-ARs in undifferentiated cells and evidence of a significant increase in staining intensity after 24 hrs treatment with bFGF alone; scale bars, 100 μm, (400× magnification);

FIG. 7 displays the influence of ARs on differentiation of MLPC into oligodendrocytes; (A) phase contrast images of cells at 30 days of differentiation, in the presence of NE, exhibited a complex multipolar morphology; (B) cells differentiated for 30 days in the medium where NE was substituted by the β-AR agonist isoproterenol and the α1-AR agonist phenylephrine; cells were morphologically comparable to cells treated with NE; (C) cells differentiated in the presence of the β-AR agonist isoproterenol frequently displayed bipolar morphology, resembling immature oligodendrocyte progenitors; (D) substitution of NE by the α1-AR agonist phenylephrine resulted in mature morphology in 10% of cells; the remainder of the cells showed only partial process development or remained flat; (E) cells differentiated for 30 days without activation of ARs by NE or AR-agonists continued to exhibit a mostly flat morphology; (F) Stimulation of both α1- and β1-ARs is required for optimal differentiation; immunocytochemical analysis of cells differentiated for 30 days in the presence of β1-AR agonist isoproterenol and the α1-AR agonist phenylephrine; cells stained positively for A2B5, O4, O1 and MBP; scale bars, 100 μm; (G) comparison of differentiation levels achieved by activation of both ARs by NE, by both AR agonists (Iso+Phen), by β-AR by isoproterenol (Iso) and by α1-AR agonist phenylephrine (Phen); error bars represent the SD; and

FIG. 8 shows a flow diagram of a method of the invention.

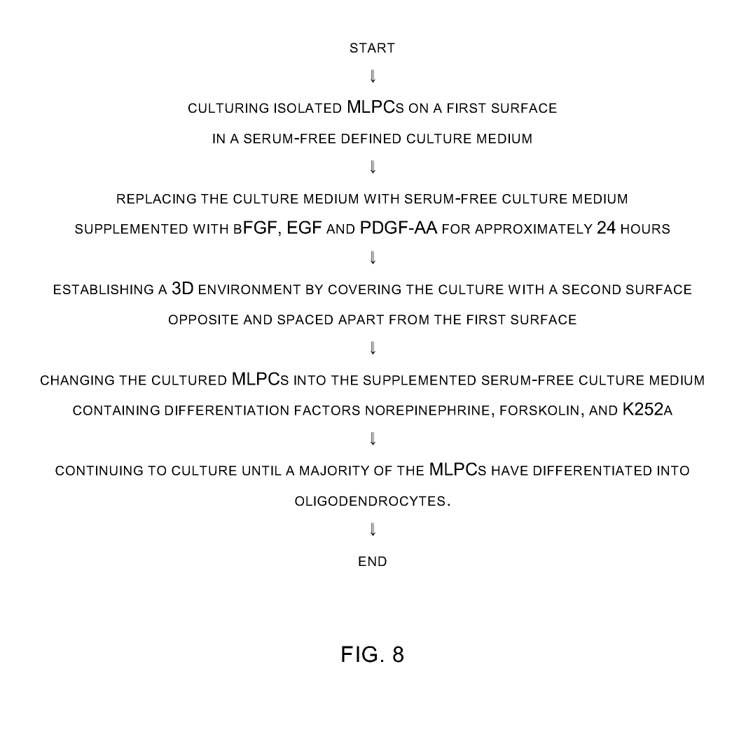

### DETAILED DESCRIPTION OF THE PREFERRED EMBODIMENT

The present invention will now be described more fully hereinafter with reference to the accompanying drawings, in which preferred embodiments of the invention are shown.

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not the only ones suitable for use in the invention.

Moreover, it should also be understood that as any measurement can be expected to have some inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Further, any publications, patent applications, patents, and other references mentioned herein are incorporated by reference in their entirety as if they were part of this specification. However, in case of conflict, the present specification, including any definitions, will control. In addition, the materials, methods and examples given are illustrative in nature only and not intended to be limiting.

Accordingly, this invention may be embodied in many different forms and should not be construed as limited to the illustrated embodiments set forth herein. Rather, these illustrated embodiments are provided so that this disclosure will be thorough, complete, and will fully convey the scope of the invention to those skilled in the art. Other features and advantages of the invention will be apparent from the following detailed description, and from the claims.

### Experimental Procedures

### Modification of Surfaces

Glass coverslips were cleaned using HCl/methanol (1:1), soaked in concentrated H₂SO₄ for 30 min then rinsed in double deionized H₂O. Coverslips were then boiled in deionized water, rinsed with acetone and oven dried. The trimethoxysilylpropyldiethylenetriamine (DETA, United Chemical Technologies), tridecafluoro-1,1,2,2-tetrahydroctyl-1-trichlorosilane (13F, Gelest) and poly(ethylene glycol) (PEG, Sigma Chemical Co., St. Louis, Mo.) monolayers were formed by the reaction of the cleaned surfaces with a 0.1% (v/v) mixture of the organosilane in toluene (Fisher T2904). The DETA coverslips were heated to just below the boiling point of toluene for 30 min, and then rinsed with toluene, reheated to just below the boiling temperature, and then oven dried. Surfaces were characterized by contact angle and X-ray photoelectron spectroscopy methods as described previously (Hickman et al., 1994).

### Cell Culture

MLPCs, human umbilical cord blood-derived clonal cell lines, passage 5, were purchased from BioE Inc. St. Paul, Minn. The cells were cultured in tissue-culture treated T-75 flasks and maintained in growth medium Dulbecco's Modified Eagle Media-high glucose (DMEM, Gibco BRL, Rockville, Md.) with the addition of 15% fetal bovine serum (Stem Cell Technologies, Vancouver, BC), and 1% Antibiotic Antimycotic (Gibco BRL) at 37° C. in humidified atmosphere containing 5% CO₂. The medium was changed every 3-4 days. Upon reaching 60% confluence, cells were passaged by trypsinization (0.05% trypsin/EDTA solution; Gibco BRL) and replated at a 1:3 ratio. Passage 8 was used for the experiments unless otherwise indicated.

### Induction of Oligodendrocyte Differentiation

MLPCs were seeded on DETA-coated glass coverslips (18 mm) at a density of 4×10³ cells/cm² in growth medium. After 3 days, at about 60% confluency, cells were incubated for 24 hours in pre-induction medium, consisting of DMEM, 15% FBS. FGF-2, EGF (20 ng/ml each, R & D Systems, Minneapolis, Minn.) and PDGF-AA (10 ng/ml, Chemicon International, Temecula, Calif.). To initiate differentiation, the pre-induction medium was removed, the cells were washed 2× with Hanks' balanced salts solution (Gibco BRL) and transferred to serum-free differentiation medium. Differentiation medium was composed of DMEM, N2 supplement (1%, Gibco BRL), 10 μM forskolin, 5 U/ml heparin (Sigma, St. Louis, Mo.), 5 nM K252a, FGF-2, EGF, PDGF-AA (10 ng/ml each) and 20 μM NE (Sigma). The differentiation medium was changed every other day, while NE was added daily. For agonist experiments, norepinephrine was substituted with either the α1-adrenoceptor agonist isoproterenol or the β-receptor agonist isoproterenol (20 μM each, Sigma).

Construction of 3D Environment MLPCs were plated on DETA-coated glass coverslips (18 mm) in growth medium. At about 60% confluence, growth medium was replaced with pre-induction medium. After the medium replacement, another set of coverslips was placed on the top of the cells. Prior to the placement, top coverslips were ethanol sterilized and washed in the pre-induction medium. After 24 hours the pre-induction medium was removed, the cells were washed 2× with Hanks' Balanced Salts Solution and transferred to serum-free differentiation medium. The differentiation medium was changed every other day and NE was added daily.

### Morphological Analysis

Phase-contrast images were taken with a commercial Nikon Coolpix 990 camera using the Zeiss Axiovert S100 microscope. Pictures were analyzed using Scion Image Software (Scion Corp., Frederick, Md.).

### Immunocytochemistry

To characterize cells by immunocytochemistry, the top coverslips, if present, were first carefully removed. Cells were then briefly washed with Hanks' balanced salts solution and fixed in 4% paraformaldehyde for about 18 min. Fixed cells were stored in PBS, permeabilized with 0.5% Triton 100× in PBS for 7 min. blocked with 5% donkey serum for 1 hr, followed by incubation with primary antibody overnight at 4° C. Primary antibodies were mouse monoclonal A2B5 (MAB312, 1:250). O4 (MAB345, 1:100). O1 (MAB344, 1:200), MBP (1:25), rabbit polyclonal anti-galactocerebroside (AB142, 1:200), all from Chemicon, mouse monoclonal beta-2-AR (sc-81577, 1:200), rabbit polyclonal beta-1AR (sc-567, 1:250) from Santa Cruz Biotechnology, and alpha-1-AR (ab3462, 1:1000, Abcam Inc. Cambridge, Mass.). Following a PBS washing, cells were incubated with an Alexa Fluor 488-conjugated anti-mouse IgG or Alexa Fluor 488-conjugated anti-rabbit IgG for 2 hours at room temperature. After a PBS washing, the coverslips were mounted with Vectashield mounting medium (H1000, Vector Laboratories, Burlingame, Calif.) onto slides. For visualizing cellular nuclei, the specimens were counterstained with DAPI. Immunoreactivity was observed and analyzed by using an Ultra VIEW™ LCI confocal imaging system (Perkin Elmer).

### Quantification

The morphological and immunocytochemical quantification was performed on undifferentiated stem cells or cells during various differentiation stages. For each coverslip, at least 10 pictures were taken from randomly chosen views under 200× magnification. All the marker-positive cells were counted, as well as the total number of cells in these views. At least three coverslips in each group were quantified and data were expressed as average±standard deviation (SD). Statistical differences between different experimental groups were analyzed by Student's t-test.

### Results

### MLPCs and Oligodendrocytes May Share Neuroepithelial Origin

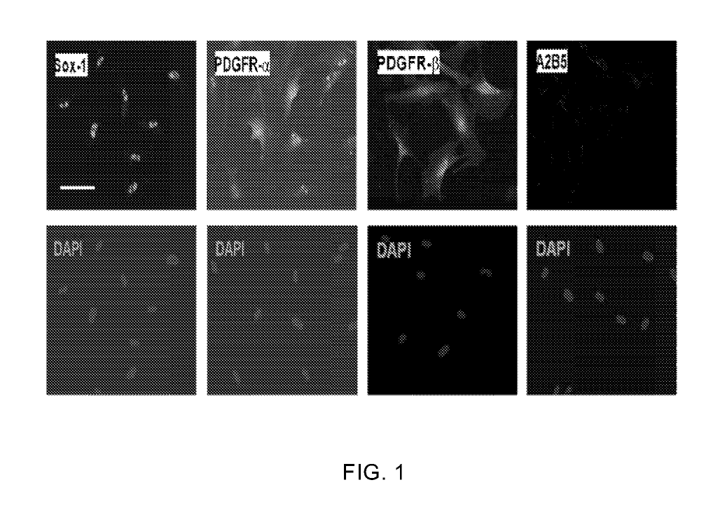

Oligodendrocytes arise from the Sox1 positive neuroepithelium during development. Induction of oligodendrocyte fate is characterized by expression of A2B5 and PDGFR-α (Behar. 2001; Pringle et al., 1996). In order to explore whether untreated MLPCs could have some neuroepithelial or oligodendrocyte progenitor characteristics, immunocytochemical analysis for expression of Sox1. A2B5 and PDGFR-α was performed. The results indicated that untreated MLPCs were Sox1 positive. This suggests that MLPCs, like oligodendrocytes, originate from the neuroepithelium. Untreated cells were PDGFR-α positive and A2B5 negative but expressed PDGFR-β (FIG. 1). The negative expression of A2B5 and positive staining for PDGFR-β (FIG. 1) distinguished the untreated MLPCs from oligodendrocyte progenitor cells. These results are interesting when compared to recently published findings demonstrating that Sox1+ neuroepithelium also gives rise to the first wave of multipotent MSCs, generated during prenatal development (Miller, 2007; Takashima et al., 2007).

MLPC Differentiation into Oligodendrocyte Lineage in a 2D Environment

The development into an oligodendrocyte phenotype is controlled by distinct molecular mechanisms. These mechanisms are influenced by various factors such as PDGF-AA, bFGF, EGF and changes in intracellular cAMP levels. There is also evidence supporting the role of NE during oligodendroglial development (Baron et al., 2000: Chandran et al., 1998; Ghiani et al., 1999: Mokry et al., 2007). Based on these previous findings, MLPCs were induced to differentiate into the initial stages of oligodendrocyte lineage in a defined, serum-free culture system. Prior to differentiation, the MLPCs were plated on trimethoxy-silylpropyl-diethylenetriamine (DETA)-coated coverslips and allowed to evenly spread and expand either for 3 days or to about 60% confluence. It was observed that higher cell densities reduced the differentiation efficiency, whereas low cell density negatively affected survival. When the cells were about 60% confluent, the culture medium was replaced with the pre-induction medium supplemented with bFGF, EGF and PDGF-AA. After 24 hrs, cells were transferred into the differentiation medium.

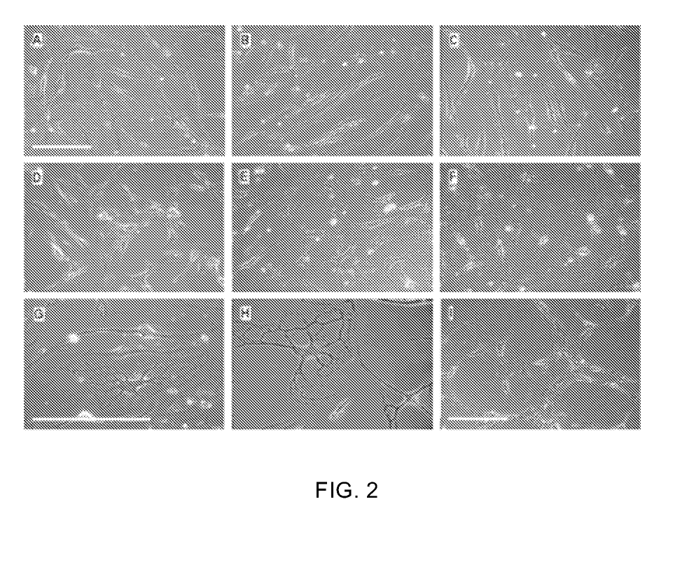

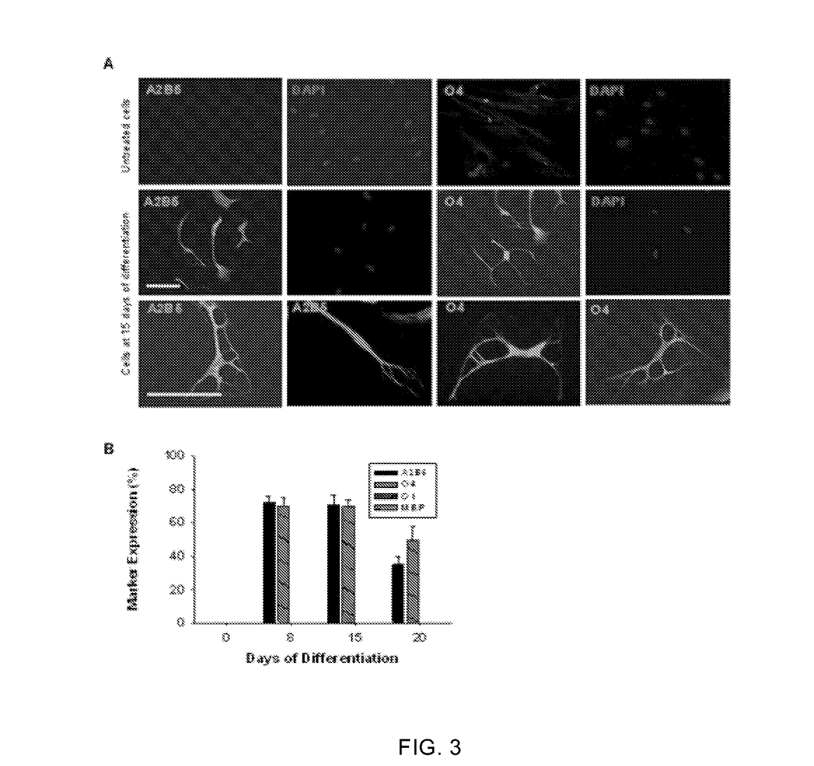

Differentiation medium contained the growth factors bFGF. EGF and PDGF-AA along with K252a, heparin, forskolin and NE. The differentiation factors were NE, forskolin and K252a appeared essential, as the desired morphology was not observed in the absence of any of these factors (FIGS. 2B, 2C, 2D). Both forskolin and K252a are factors frequently used during different stages of stem cell differentiation; however norepinephrine emerged as the novel stem cell differentiation factor that uniquely promoted the MLPCs along an oligodendrocyte lineage. Absence of the growth factors increased the differentiation rate but resulted in decreased survival and less elaborate process formation (FIG. 2E). After the transfer into the differentiation medium, the MLPCs exhibited cell shape changes, from that of a fibroblast morphology (FIG. 2A) to refractile cell bodies. Within 8 days of differentiation, approximately 70% of cells developed multiple processes and FIGS. 2F, 2G, 2H reflect the morphology development at day 15. During the process, a close correlation between the passage number and the differentiation potential was observed. The most favorable outcome for differentiation of the MLPCs was found when utilizing cells from passage 8. Earlier passages did not respond as well to the treatment and retained higher proliferation rates. Later passages exhibited a somewhat decreased differentiation capacity and the propensity towards senescence. Immunocytochemical analysis was performed using the antibodies for specific stages of oligodendrocyte differentiation (FIGS. 3A, 3B). The untreated MLPCs showed negative staining for A2B5 and faint staining for O4. Cells were also negative for the more mature oligodendrocyte markers O1 galactocerebroside and MBP. However, at 8 days of differentiation, 72.4±3.4% of cells exhibited positive staining for A2B5 and 69.9±4.9% for O4, but expression of O1 galactocerebroside and MBP was absent at this time period in the 2D environment.

These results indicate that in response to the treatment, the majority of MLPCs acquired cellular characteristics of immature oligodendrocyte precursor cells. The expression of A2B5 and O4, accompanied by a multi-process morphology, persisted to day 15, but the precursors did not achieve a fully differentiated oligodendrocyte phenotype. After 15 days of differentiation, cells began to lose their multi-process morphology and became mostly bipolar and spindle shaped (FIG. 21). At day 20, 35.0±4.8% of cells remained A2B5 positive, 49.7±7.9% O4 positive and O1, MBP negative (FIG. 3B). In addition, limited cell survival was observed after day 20 in culture.

### MLPCs Differentiation in a 3D Environment

Because it has been shown to be an important feature in previous studies of cellular development, we examined the possible effect of a simple 3D environment on oligodendrocyte lineage progression. To construct this 3D environment, cells were differentiated between 2 coverslips. Initially, undifferentiated cells were plated onto DETA-coated coverslips at the bottom of 12-well plates. When the cells reached approximately 60% confluence, the culture medium was replaced with the pre-induction medium, then, after the medium replacement, an unmodified glass coverslip was placed over the top of the cultured cells.

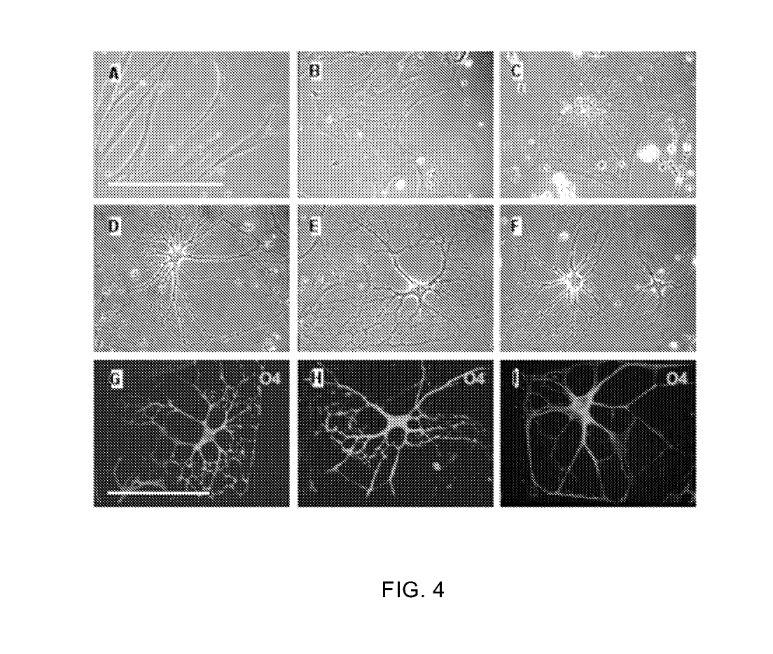

In the 3D environment, within 24 hrs, significant cell morphological flattening and spreading was observed (FIG. 4B). After 24 hrs, the pre-induction medium was replaced with differentiation medium and there was a further increase in cell flattening (FIG. 4C). Within 10 days of differentiation cells began to form processes and the cell bodies slowly contracted. Process development and branching continued for 3 weeks. After 30 days of differentiation, approximately 85% of the cells had elaborated an extensive network of processes (FIGS. 4D, 4E, 4F). The presence of PDGF was required for process formation, as in its absence cells progressed through initial differentiation stages but lost their multi-process morphology after 2 weeks of differentiation. The presence of bFGF and EGF was not essential but resulted in increased branching and the development of highly elaborated processes (FIGS. 4G, 4H, 4I).

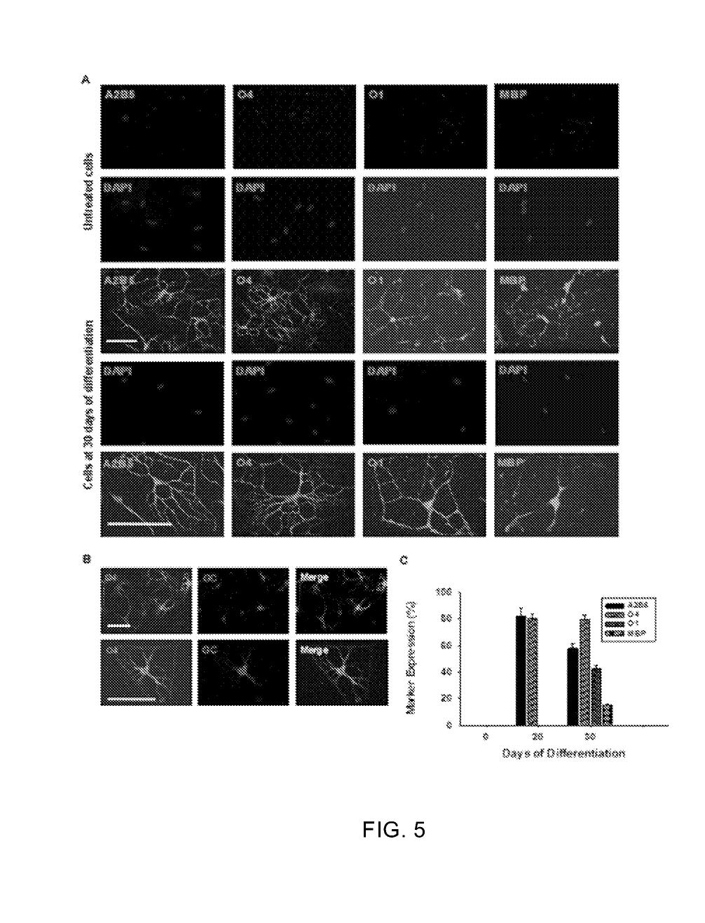

Immunocytochemical analysis revealed that after 20 days of differentiation approximately 81.8±6.6% of cells expressed the oligodendroglial marker A2B5 and about 80.6±2.9% expressed O4 (FIG. 5C). At 30 days of differentiation, 57.7±3.6% of the cells stained positively for A2B5, 79.6±2.9% for O4, 42.1±2.7% for the committed oligodendrocyte marker O1 galactocerebroside and 15.2±0.5% for MBP (FIGS. 5A, 5B, 5C). The 3D environment appeared to play an important role in differentiation, oligodendrocyte commitment and lineage progression. There was decreased cell proliferation and, unlike in the 2D environment, passage numbers did not significantly affect differentiation in the 3D environment. Even after the removal of NE from the differentiation medium after 20 days, the cells retained their differentiated morphology for an additional 10 days in culture.

The contribution of the surface chemistry of the top coverslip was also qualitatively investigated, as previously it has been shown that surface composition can have a dramatic effect on cellular response and differentiation (Ravenscroft-Chang et al., 2010; Spargo et al., 1994; Stenger et al., 1993). To determine the most appropriate 3D conditions for differentiation, the top glass coverslips were also modified with various surface chemistries which had been found previously to selectively promote or repel cell adhesion (Table 1). Unmodified glass coverslips were used as a control. To promote cell adhesion, the top coverslip was coated with a DETA monolayer. This environment, in which cells were attached to both top and bottom coverslips, produced initially good differentiation but eventually caused increased cell death, possibly due to damage from cell movement during feeding and morphological evaluation as the cells were well adhered to both surfaces. For the inverse situation the top coverslip was coated with polyethyleneglycol (PEG) or with a non-adhesive fluorinated silane (13F) monolayer. The PEG coated top coverslips resulted in good cell survival but a lesser degree of differentiation. 13F coated coverslips (contact angle >100°) triggered significant cell death. Thus it was determined that the glass coverslips controls were the most suitable top surfaces for optimal differentiation, as the cells did not adhere to the glass, and remained on the bottom DETA coated coverslips even after the top coverslip was removed.

### MLPCs Express Functional ARs in the 3D System

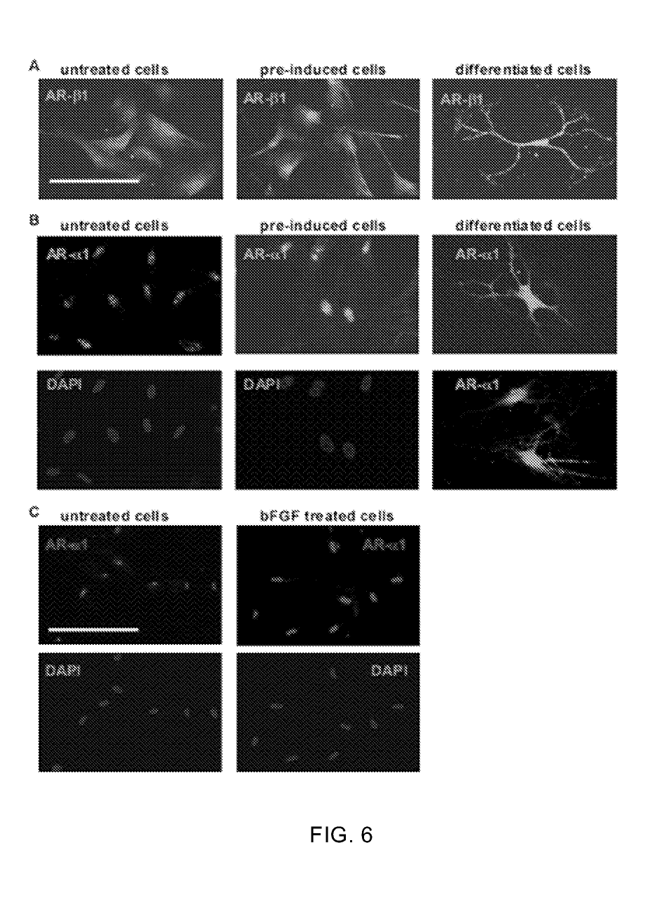

To investigate the role of adrenergic signaling mechanisms in oligodendrocyte differentiation from the MLPCs, immunocytochemical analysis was performed for expression of α- and β-ARs. The findings indicated that MLPCs already expressed β1-ARs on the cell surface before differentiation (FIG. 6A). No expression of β2-ARs before or during differentiation was detected. Expression of α1-ARs was first observed at the nucleus and the intensity of staining significantly increased after treatment with pre-induction medium supplemented with bFGF, EGF and PDGF-AA (FIG. 6B). Further analysis revealed that bFGF alone was able to increase nuclear expression of α1-ARs (FIG. 6C) in a time and dose dependent manner (results not shown). At day 15 of differentiation the α1-ARs began to relocate to the cell surface. At day 30, differentiated cells expressed α1-ARs at the surface of cell bodies and to a lesser degree in the processes (FIG. 6B). This surface expression was observed only in cells with a multi-process morphology, whereas in cells exhibiting a flat morphology, or undifferentiated cells, the α1-ARs remained at the nucleus. These studies are consistent with recent findings demonstrating nuclear localization of α1-AR (Huang et al., 2007; Wright et al., 2008). The published studies provided a new model for α1-AR signaling, in which a signal is transduced from the nucleus to the plasma membrane, and is confirmed in this cellular transformation as well. The same initial expression patterns were noted in the 2D system, but the cells were not viable past day 20.

### Activation of Both α1- and β1-AR is Needed for Differentiation in the 3D System

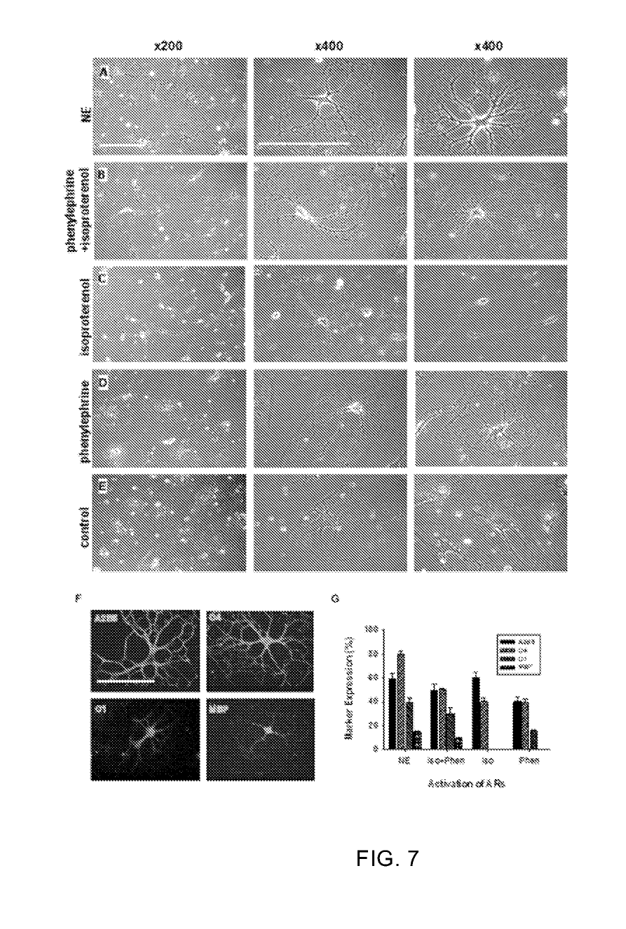

To assess the role of each adrenergic receptor in the differentiation process, NE was substituted in the differentiation medium with equimolar concentrations of the β-AR agonist isoproterenol, the α1-AR agonist phenylephrine or with both agonists. Daily treatment with isoproterenol induced morphological changes, cell body contraction and formation of processes within the first 15 days of treatment. However, approximately 60% of the cells displayed a bipolar morphology resembling immature oligodendrocyte progenitors. Cells did not change their bipolar morphology within 30 days of differentiation (FIG. 7C) and exhibited enhanced cell death. In order to characterize these cells, immunocytochemical analysis at day 30 of differentiation was done. We observed that 57.9±4.9% of the cells expressed A2B5 and 42.5±2.7% expressed O4, however the cells were O1 and MBP negative (FIG. 7G). These results demonstrated that stimulation of β-AR by isoproterenol induced the initial stages of differentiation. However, β-AR treatment alone was not sufficient to direct the MLP cells into a more mature stage of differentiation.

Daily treatment of the MLPCs with the α1-AR agonist phenylephrine initially resulted in only modest effects. Good cell survival was observed but the majority of cells maintained a flat morphology. Within 15 days of differentiation approximately 40% of the cells began to develop processes, however at day 30, only 15% of the cells exhibited more mature morphology with developed processes. The majority of cells showed only partial process development and branching or maintained a flat morphology (FIG. 7D). Immunocytochemical analysis at day 30 revealed that 40.5±3.4% of cells stained positively for A2B5, 39.8±2.8% for O4 and 15.2±1.5% for O1 (FIG. 7G). The results suggested that activation of α1-AR could play a role in more advanced stages of differentiation in which cells start to lose expression of A2B5 and begin to express O1.

Daily treatments of cells with both isoproterenol and phenylephrine resulted in formation of multiple processes and the treated cells became morphologically similar to those treated with NE (FIGS. 7A and 7B). At day 30 of the differentiation period, 48.7±4.9% of the cells stained positively for A2B5, 50.2±1.1% for O4, 28.9±5.2% for O1 and 9.7±0.7% for MBP (FIGS. 7F, 7G).

The results indicate that stimulation of both the α1- and β-ARs is required for optimal differentiation. This suggests a close interplay between both ARs, ultimately resulting in the expression of genes essential for oligodendrocyte development.

### Discussion

Oligodendrocytes, like most other cells in the CNS, arise from Sox-1 positive neuroepithelial cells of the neural tube (LeVine and Goldman, 1988; Noll and Miller, 1993; Warf et al., 1991). In this study, oligodendrocytes were generated from Sox-1 positive MLPCs from human umbilical cord. It is possible that MLPCs, like cells of the CNS and early waves of multipotent MSCs, originate from neuroepithelium during development (Miller, 2007; Takashima et al., 2007). MLPCs from umbilical cord are collected at birth and have the potential to give rise to all three embryonic layers (van de Ven et al., 2007). This study has indicated that MLPCs display extreme sensitivity to their environment. Their fate depends not only on soluble factors but also on the surrounding physical cues. The combination of these external signals, processed through signal transduction networks, altered the cell morphology and fate decision to differentiate along the oligodendrocyte linage.

Electron microscopy studies have provided evidence for direct noradrenergic control of the oligodendroglia) and astroglial cells throughout the cortex (Paspalas and Papadopoulos, 1996). Oligodendrocytes were the major target of the noradrenergic fibers, exhibiting a light thickening at the sites of contact. It was reported that oligodendrocytes expressed α1 and β-ARs and their activation by NE accelerated differentiation of the oligodendrocyte precursors (Ghiani et al., 1999; Khorchid et al., 2002; Ventimiglia et al., 1987). In spite of this, there are no known studies using NE as a key factor to induce differentiation of stem cells into oligodendrocytes. To explore this possibility, MLPCs were analyzed for expression of ARs, and it was found that the undifferentiated cells expressed both α1-ARs and β1-ARs. The β1-ARs were localized on the surface before and during the differentiation. Surprisingly, we did not observe typical surface expression of the α1-ARs; instead, the these were localized at the nucleus. The intensity of nuclear staining significantly increased after treatment with bFGF. However, as the cells exhibited a more differentiated phenotype after 15 days of differentiation, relocation of α1-ARs to the surface was observed. Nuclear localization of α1-ARs is consistent with a recently proposed model for α1-AR signaling in cardiac myocytes. In this new model, activation of α1-AR signaling is initiated at the nuclear membrane and results in localization of activated ERK in calveolae at the plasma membrane (Huang et al., 2007; Wright et al., 2008).

Differentiation was initiated by the transfer of MLPCs into the differentiation medium in a 2D environment. The differentiation medium contained NE along with forskolin, K252a, heparin, PDGF-AA, bFGF and EGF. Within 8 days in the differentiation medium process formation was observed and immunocytochemical analysis indicated a positive reactivity to A2B5 and O4 primary antibodies. In spite of this, cells did not progress further along the oligodendrocyte lineage. After 2 weeks in differentiation medium, cells exhibited bipolar and spindle like morphology and remained A2B5 and O4 positive but O1 negative, and prolonged differentiation time also significantly increased cell death.

A 3D microenvironment was constructed to combine chemical and physical cues shown to influence lineage commitment during development in other systems. The MLPCs responded to the 3D environment initially by cell flattening and later, within 2 weeks of differentiation, formation of processes. At 30 days, 42.1±2.7% of cells expressed the O1 antigen, indicating terminally differentiated oligodendrocytes, and 15.2±0.5% of the cells expressed MBP with increased cell survival. The differentiated cells survived for more than 40 days in culture. Importantly, the oligodendrocytes retained their differentiated state even after removal of the NE after 20 days.

It is well established in other systems that after removing growth factors from the medium, oligodendrocyte precursors exit cell cycle, stop dividing and terminally differentiate (Izrael et al., 2007; Nguyen et al., 2006; Rogister et al., 1999). In our system more complex branching, process development and increased survival was demonstrated in the presence of growth factors. This could be explained by the combined effect of forskolin and norepinephrine. Both factors are known to increase cAMP levels, and increased cAMP levels inhibit proliferation of oligodendrocyte precursors (Ghiani et al., 1999; Raible and McMorris, 1989). Thus, the removal of growth factors was not essential for cell cycle exit and terminal differentiation. Decreased proliferation with increased cell flattening and spreading was also observed after the introduction of the top coverslip.

It has also been demonstrated previously that stimulation of β-ARs induce differentiation through an increase in intracellular CAMP and through the activation of proteins known to be involved in cell cycle arrest (Ghiani et al., 1999). There are also studies revealing that p38MAPK and Erk1/2 have roles in differentiation of oligodendrocyte progenitors (Bhat et al., 2007). Both ARs can activate p38MAPK while Erk1/2 is a downstream target of α1-AR. It was shown in these studies that p38MAPK activity was required for the progression of bipolar early progenitors (A2B5+, O4−) to multipolar late progenitors (O4+, O1−), and that Erk1/2 activity was necessary for progression of late progenitors to oligodendrocytes (Baron et al., 2000).

Our results, however, indicated that the majority of cells treated with the β-AR agonist isoproterenol remained bipolar even after 30 days of differentiation. Immunocytochemical analysis indicated differentiation arrest at the stage where A2B5+ cells begin to express O4, perhaps due to insufficient activation of Erk1/2 signaling. In contrast, cells treated with the α1-AR agonist phenylephrine showed higher survival and after 30 days of differentiation 15.2±1.5% of cells had progressed to the O1+ stage, possibly due to increased stimulation of the Erk1/2 signaling pathway. However, simultaneous activation of both receptors by NE, or by both agonists, was the best strategy, possibly by supplying the optimal cAMP levels and stimulating essential signaling pathways engaged in the close interplay during differentiation.

The present study demonstrates the significance of the cellular microenvironment as a driving aspect in human stem cell differentiation. A 3D environment was constructed and a novel small molecule was utilized to induce differentiation of MLPCs, whereas neither condition alone produced functional differentiation. The mechanical cues in combination with soluble factors influenced the progression of MLPCs along the oligodendrocyte lineage.

The herein disclosed method of generating terminally differentiated functional human oligodendrocytes will be useful in providing a supply of those cells for study and for treating demyelinating conditions such as multiple sclerosis, neuropathy and in traumatic brain injury.

Accordingly, in the drawings and specification there have been disclosed typical preferred embodiments of the invention and although specific terms may have been employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

### REFERENCES

Asotra, K., and Macklin, W. B. (1993). Protein kinase C activity modulates myelin gene expression in enriched oligodendrocytes. J Neurosci Res 34, 571-588.
Bansal, R., and Pfeiffer, S. E. (1992). Novel stage in the oligodendrocyte lineage defined by reactivity of progenitors with R-mAb prior to O1 anti-galactocerebroside. J Neurosci Res 32, 309-316.
Baron, W., Metz, B., Bansal, R., Hoekstra, D., and de Vries, H. (2000). PDGF and FGF-2 signaling in oligodendrocyte progenitor cells: regulation of proliferation and differentiation by multiple intracellular signaling pathways. Mol Cell Neurosci 15, 314-329.
Behar, T. N. (2001). Analysis of fractal dimension of O2A glial cells differentiating in vitro. Methods 24, 331-339.
Bernstein, M., Lyons, S. A., Moller, T., and Kettenmann, H. (1996). Receptor-mediated calcium signalling in glial cells from mouse corpus callosum slices. J Neurosci Res 46, 152-163.
Bettinger, C. J., Langer, R., and Borenstein, J. T. (2009). Engineering substrate topography at the micro- and nanoscale to control cell function. Angew Chem Int Ed Engl 48, 5406-5415.
Bhat, N. R., Zhang, P., and Mohanty, S. B. (2007). p38 MAP kinase regulation of oligodendrocyte differentiation with CREB as a potential target. Neurochem Res 32, 293-302.
Bunge, M. B., Bunge, R. P., and Pappas, G. D. (1962). Electron microscopic demonstration of connections between glia and myelin sheaths in the developing mammalian central nervous system. J Cell Biol 12, 448-453.
Bunge, R. P. (1968). Glial cells and the central myelin sheath. Physiol Rev 48, 197-251.
Burdick, J. A., and Vunjak-Novakovic, G. (2008). Review: Engineered Microenvironments for Controlled Stem Cell Differentiation. Tissue Eng Part A.
Chandran, S., Svendsen, C., Compston, A., and Scolding, N. (1998). Regional potential for oligodendrocyte generation in the rodent embryonic spinal cord following exposure to EGF and FGF-2. Glia 24, 382-389.
Cohen, R. I., and Almazan, G. (1993). Norepinephrine-stimulated PI hydrolysis in oligodendrocytes is mediated by alpha 1A-adrenoceptors. Neuroreport 4, 1115-1118.
Curtis, R., Cohen, J., Fok-Seang, J., Hanley, M. R., Gregson, N. A., Reynolds, R., and Wilkin, G. P. (1988). Development of macroglial cells in rat cerebellum. I. Use of antibodies to follow early in vivo development and migration of oligodendrocytes. J Neurocytol 17, 43-54.
Das, M., Bhargava, N. Gregory, C., Riedel, L., Molnar, P., and Hickman, J. J. (2005). Adult rat spinal cord culture on an organosilane surface in a novel serum-free medium. in vitro Cell Dev Biol Anim 41, 343-348.
Engler, A. J., Sen, S., Sweeney, H. L., and Discher, D. E. (2006). Matrix elasticity directs stem cell lineage specification. Cell 126, 677-689.
Fisher, O. Z., Khademhosseini, A. Langer, R., and Peppas, N. A. (2009). Bioinspired Materials for Controlling Stem Cell Fate. Acc Chem. Res.
Ghiani, C. A., Eisen, A. M., Yuan, X., DePinho, R. A., McBain, C. J., and Gallo, V. (1999). Neurotransmitter receptor activation triggers p27(Kip1) and p21(CIP1) accumulation and G1 cell cycle arrest in oligodendrocyte progenitors. Development 126, 1077-1090.
Hickman, J. J., Bhatia, S. K., Quong, J. N., Shoen, P., Stenger, D. A., Pike, C. J., and Cotman, C. W. (1994). Rational Pattern Design for in-Vitro Cellular Networks Using Surface Photochemistry. J Vac Sci Technol A-Vac Surf Films 12, 607-616.
Hirano, A. (1968). A confirmation of the oligodendroglial origin of myelin in the adult rat. J Cell Biol 38, 637-640.
Hu, B. Y., Du. Z. W., Li, X. J., Ayala, M., and Zhang, S. C. (2009). Human oligodendrocytes from embryonic stem cells: conserved SHH signaling networks and divergent FGF effects. Development 136, 1443-1452.
Huang, Y., Wright, C. D., Merkwan, C. L., Baye, N. L., Liang, Q., Simpson, P. C. and O'Connell, T. D. (2007). An alpha1A-adrenergic-extracellular signal-regulated kinase survival signaling pathway in cardiac myocytes. Circulation 115, 763-772.
Izrael, M., Zhang, P., Kaufman, R., Shinder. V., Ella, R., Amit, M., Itskovitz-Eldor, J., Chebath, J., and Revel, M. (2007). Human oligodendrocytes derived from embryonic stem cells: Effect of noggin on phenotypic differentiation in vitro and on myelination in vivo. Mol Cell Neurosci 34, 310-323.
Kessaris, N., Pringle, N., and Richardson. W. D. (2008). Specification of CNS glia from neural stem cells in the embryonic neuroepithelium. Philos Trans R Soc Lond B Biol Sci 363, 71-85.
Khorchid, A., Cui, Q., Molina-Holgado, E., and Almazan, G. (2002). Developmental regulation of alpha 1A-adrenoceptor function in rat brain oligodendrocyte cultures. Neuropharmacology 42, 685-696.
Khorchid, A., Larocca, J. N., and Almazan, G. (1999). Characterization of the signal transduction pathways mediating noradrenaline-stimulated MAPK activation and c-fos expression in oligodendrocyte progenitors. J Neurosci Res 58, 765-778.
Levenberg, S., Huang, N. F., Lavik, E., Rogers, A. B., Itskovitz-Eldor, J., and Langer, R. (2003). Differentiation of human embryonic stem cells on three-dimensional polymer scaffolds. Proc Natl Acad Sci USA 100, 12741-12746.
LeVine, S. M., and Goldman, J. E. (1988). Embryonic divergence of oligodendrocyte and astrocyte lineages in developing rat cerebrum. J Neurosci 8, 3992-4006.
Liu, S., Qu, Y., Stewart, T. J., Howard, M. J., Chakrabortty, S., Holekamp, T. F., and McDonald, J. W. (2000). Embryonic stem cells differentiate into oligodendrocytes and myelinate in culture and after spinal cord transplantation. Proc Natl Acad Sci USA 97, 6126-6131.
Luo, Y., Kobler, J. B., Zeitels, S. M. and Langer, R. (2006). Effects of growth factors on extracellular matrix production by vocal fold fibroblasts in 3-dimensional culture. Tissue Eng 12, 3365-3374.
McBeath, R., Pirone, D. M., Nelson, C. M., Bhadriraju, K., and Chen, C. S. (2004). Cell shape, cytoskeletal tension, and RhoA regulate stem cell lineage commitment. Dev Cell 6, 483-495.
Miller. F. D. (2007). Riding the waves: neural and normeural origins for mesenchymal stem cells. Cell Stem Cell 1, 129-130.
Mokry, J., Karbanova, J., Cizkova. D. Pazour, J., Filip, S., and Osterreicher, J. (2007). Differentiation of neural stem cells into cells of oligodendroglial lineage. Acta Medica (Hradec Kralove) 50, 35-41.
Nguyen, L., Borgs, L., Vandenbosch, R., Mangin, J. M., Beukelaers, P., Moonen, G., Gallo, V., Malgrange, B., and Belachew, S. (2006). The Yin and Yang of cell cycle progression and differentiation in the oligodendroglial lineage. Ment Retard Dev Disabil Res Rev 12, 85-96.
Nistor, G. I., Totoiu, M. O. Hague, N., Carpenter, M. K., and Keirstead, H. S. (2005). Human embryonic stem cells differentiate into oligodendrocytes in high purity and myelinate after spinal cord transplantation. Glia 49, 385-396.
Noll, E., and Miller, R. H. (1993). Oligodendrocyte precursors originate at the ventral ventricular zone dorsal to the ventral midline region in the embryonic rat spinal cord. Development 118, 563-573.
Orentas, D. M., and Miller, R. H. (1998). Regulation of oligodendrocyte development. Mol Neurobiol 18, 247-259.
Paspalas, C. D., and Papadopoulos, G. C. (1996). Ultrastructural relationships between noradrenergic nerve fibers and non-neuronal elements in the rat cerebral cortex. Glia 17, 133-146.
Peters. A. (1964). Observations on the Connexions between Myelin Sheaths and Glial Cells in the Optic Nerves of Young Rats. J Anat 98, 125-134.
Pfeiffer, S. E., Warrington, A. E., and Bansal, R. (1993). The oligodendrocyte and its many cellular processes. Trends Cell Biol 3, 191-197.
Pringle, N. P., Yu, W. P., Guthrie, S., Roelink, H., Lumsden, A., Peterson, A. C., and Richardson, W. D. (1996). Determination of neuroepithelial cell fate: induction of the oligodendrocyte lineage by ventral midline cells and sonic hedgehog. Dev Biol 177, 30-42.
Raible, D. W., and McMorris, F. A. (1989). Cyclic AMP regulates the rate of differentiation of oligodendrocytes without changing the lineage commitment of their progenitors. Dev Biol 133, 437-446.
Ravenscroft-Chang, M. S. Stohlman, J. M., Molnar, P., Natarajan, A. Canavan, H. E., Teliska, M., Stancescu, M., Krauthamer, V., and Hickman, J. J. Altered calcium dynamics in cardiac cells grown on silane-modified surfaces. Biomaterials 31, 602-607.
Rogister, B., Ben-Hur, T., and Dubois-Dalcq, M. (1999). From neural stem cells to myelinating oligodendrocytes. Mol Cell Neurosci 14, 287-300.
Rosenberg, S. S., Kelland, E. E. Tokar, E., De la Torre, A. R., and Chan, J. R. (2008). The geometric and spatial constraints of the microenvironment induce oligodendrocyte differentiation. Proc Natl Acad Sci USA 105, 14662-14667.
Schaffner, A. E., Barker. J. L. Stenger, D. A., and Hickman, J. J. (1995). Investigation of the factors necessary for growth of hippocampal neurons in a defined system. J Neurosci Methods 62, 111-119.
Spargo, B. J., Testoff, M. A., Nielsen, T. B. Stenger, D. A., Hickman, J. J., and Rudolph, A. S. (1994). Spatially controlled adhesion, spreading, and differentiation of endothelial cells on self-assembled molecular monolayers. Proc Natl Acad Sci USA 91, 11070-11074.
Stenger, D. A., Pike, C. J., Hickman, J. J., and Cotman, C. W. (1993). Surface determinants of neuronal survival and growth on self-assembled monolayers in culture. Brain Res 630, 136-147.
Sternberger, N. H., del Cerro, C., Kies. M. W., and Herndon, R. M. (1985). Immunocytochemistry of myelin basic proteins in adult rat oligodendroglia. J Neuroimmunol 7, 355-363.
Takashima, Y., Era, T., Nakao, K., Kondo. S., Kasuga, M., Smith, A. G. and Nishikawa, S. (2007). Neuroepithelial cells supply an initial transient wave of MSC differentiation. Cell 129, 1377-1388.
van de Ven, C., Collins, D., Bradley, M. B., Morris, E., and Cairo, M. S. (2007). The potential of umbilical cord blood multipotent stem cells for nonhematopoietic tissue and cell regeneration. Exp Hematol 35, 1753-1765.
Varghese, K., Das, M., Bhargava, N. Stancescu, M., Molnar, P., Kindy, M. S., and Hickman, J. J. (2009). Regeneration and characterization of adult mouse hippocampal neurons in a defined in vitro system. J Neurosci Methods 177, 51-59.
Vartanian, T., Sprinkle, T. J., Dawson, G., and Szuchet, S. (1988). Oligodendrocyte substratum adhesion modulates expression of adenylate cyclase-linked receptors. Proc Natl Acad Sci USA 85, 939-943.
Ventimiglia, R., Greene, M. I., and Geller, H. M. (1987). Localization of beta-adrenergic receptors on differentiated cells of the central nervous system in culture. Proc Natl Acad Sci USA 84, 5073-5077.
Vogel, V., and Sheetz, M. (2006). Local force and geometry sensing regulate cell functions. Nat Rev Mol Cell Biol 7, 265-275.
Volpe, J. J. (2008). Neurology of the Newborn, 5 edn (Elsevier Health Sciences).
Warf, B. C., Fok-Seang, J., and Miller, R. H. (1991). Evidence for the ventral origin of oligodendrocyte precursors in the rat spinal cord. J Neurosci 11, 2477-2488.
Wright, C. D., Chen, Q., Baye, N. L., Huang, Y., Healy, C. L., Kasinathan, S., and O'Connell, T. D. (2008). Nuclear alpha1-adrenergic receptors signal activated ERK localization to caveolae in adult cardiac myocytes. Circ Res 103, 992-1000.

TABLE I
Percentage of cells developing processes in
response to top coverslip modification.

| | Top Coverslip Modification | | | |
|---|---|---|---|---|
| Development of | | | | Unmodified |
| processes (%) | DETA | PEG | 13F | Glass |
| Day 20 | 66.9 ± 2.8 | 53.2 ± 1.3 | 38.1 ± 3.1 | 77.6 ± 2.2 |
| Day 30 | 69.2 ± 10.4 | 55.5 ± 8.2 | ** | 85.0 ± 2.1 |

Data show mean ± SD for three coverslips at day 20 and 30 of differentiation.
** No surviving cells were observed at day 30.

That which is claimed:

1. A method of producing oligodendrocytes by in vitro differentiation of human multi-potent progenitor cells (MLPCs), the method comprising:
culturing isolated MLPCs on a first surface in a serum-free defined culture medium;
replacing the culture medium with serum-free culture medium supplemented with bFGF, EGF and PDGF-AA for approximately 24 hours;
establishing a 3D environment by covering the culture with a second surface opposite and spaced apart from the first surface, so as to contain the MLPCs therebetween;
changing the cultured MLPCs into the supplemented serum-free culture medium further supplemented with differentiation factors norepinephrine, forskolin, and K252a; and
continuing to culture until a majority of the MLPCs have differentiated into oligodendrocytes.

2. The method of claim 1, wherein said first surface comprises a pre-treated sterile surface.

3. The method of claim 1, wherein said surface comprises a DETA-coated glass surface.

4. The method of claim 1, wherein culturing is continued until the MLPCs reach approximately 60% confluence.

5. The method of claim 1, wherein establishing the 3D environment is concurrent with the replacing step.

6. Isolated oligodendrocytes produced according to the method of claim 1.

7. A method of treatment for a subject afflicted by a disease characterized by central or peripheral nervous system deficit, the method comprising transplanting into the subject oligodendrocytes produced according to the method of claim 1.

8. The method of claim 7, wherein the deficit comprises demyelination.

9. A method of producing oligodendrocytes in vitro, the method comprising:
culturing human MLPCs within a three-dimensional environment in a defined serum-free growth medium having N2, forksolin, heparin, K252a, FGF-2, EGF, PDGF-AA and norepinephrine.

10. The method of claim 9, wherein the three-dimensional environment is contained between opposing and spaced apart DETA monolayers.

11. The method of claim 10, wherein the DETA monolayers are supported on glass surfaces.

12. The method of claim 9, wherein the defined serum-free medium is replaced about every other day.

13. The method of claim 9, wherein the norepinephrine is replenished daily.

14. The method of claim 9, wherein culturing continues until a majority of the MLPCs have differentiated into oligodendrocytes.

15. The method of claim 14, wherein the oligodendrocytes exhibit one or more surface antigens indicative of maturation.

16. A method of treatment for a subject afflicted by a disease characterized by central or peripheral nervous system deficit, the method comprising transplanting into the subject oligodendrocytes produced according to the method of claim 9.

17. The method of claim 16, wherein the deficit comprises demyelination.

18. A method of producing oligodendrocytes in vitro, the method comprising:
culturing human MLPCs within a three-dimensional environment in a defined serum-free growth medium and sufficiently stimulating adrenergic pathways in the MLPCs so as to induce their differentiation into oligodendrocytes.
